(19) United States
(12) Patent Application Publication
(10) Pub. No.: US 2023/0000374 A1
(43) Pub. Date: Jan. 5, 2023

(54) SLEEVE PART AND MEASURING DEVICE

(71) Applicant: PULSION MEDICAL SYSTEMS SE, Feldkirchen (DE)

(72) Inventors: Aaron Weber, Markt Schwaben (DE); André Hein, Esslingen am Neckar (DE)

(73) Assignee: PULSION MEDICAL SYSTEMS SE, Feldkirchen (DE)

(21) Appl. No.: 17/780,948

(22) PCT Filed: Nov. 30, 2020
(86) PCT No.: PCT/EP2020/083969

§ 371 Date: Aug. 10, 2022

(30) Foreign Application Priority Data
Dec. 1, 2019 (DE) 102019008331.8

(51) Int. Cl. A61B 5/022; A61B 5/0225; A61B 5/00

(52) U.S. Cl. CPC A61B 5/02241; A61B 5/02255; A61B 5/702; A61B 2560/0443; A61B 2562/12

(57) ABSTRACT

The two inflatable sleeve cushions (10) which can be arranged in the receiving tubes (9) are each connected by means of a connection (11) at the interface between sleeve part (2) and base part (3) to the pressure generation and pressure control system in the base part (3). A valve device is preferably provided on the connection (11). The channels (19) that connect the respective connection (11) to the respective sleeve cushion (20) are formed in the main body (21) of the receiving body, said main body being produced from plastic by means of injection moulding, and are covered towards the outside by means of a cover (20). The connections (11) are disposed one behind the other in the direction parallel to the axial direction of the receiving tubes (9). From there, each channel (19) is first guided upwards, then diagonally to the side, and finally downwards to the fluid access opening (22) of the respective sleeve cushion (10).

### TECHNICAL FIELD

The present invention relates to a cuff part, in particular for a non-invasive blood pressure measuring device, and to a measuring device for parallel or alternating continuous determination of intra-arterial blood pressure on at least two fingers of a hand

### PRIOR ART

The (in particular arterial) blood pressure of a patient is one of the most important measured variables in medical technology, and known, in particular also non-invasive, measuring technology associated with this is extremely diverse. This applies above all to measuring technology for continuously monitoring blood pressure over a prolonged period of time, for example in intensive care medicine, but also in emergency medicine and during surgical interventions.

For reasons of good accessibility, the blood pressure measuring device is often attached to a limb of a patient, for example an applanation tonometric sensor of the radial artery on the forearm or a finger sensor operated in a photoplethysmographic manner according to the so-called “Vascular Unloading Technique” according to Penaz. Such pressure measuring devices are known, for example, from U.S. Pat. No. 4,406,289, U.S. Pat. No. 4,524,777, U.S. Pat. No. 4,726,382, WO 2010/050798 A1, WO 2000/059369 A1, WO 2011/045138 A1, WO 2011/051819 A1, WO 2011/051822 A1, WO 2012/032413 A1, and WO 2017/143366 A1.

In the Vascular Unloading Technique, near-infrared light is radiated into a finger and the pulsatile (pulse-shaped) blood flow (actually the changing blood volume) in the finger is determined from the non-absorbed portion captured by means of a photodetector. For this process, also known as photoplethysmography (PPG), the (near-infrared) light is usually generated using one or more light-emitting diodes (LEDs), which work with one or more wavelengths, and is detected using one or more light-sensitive receiver diodes (photodiodes). Other types of photoreceivers besides diodes are also suitable in principle.

A control system now keeps constant the plethysmographically registered flow (or the detected blood volume) and thus the resulting photoplethysmographic signal (volume signal v(t)) by applying counterpressure in a cuff (cuff pressure) pc(t) on the finger. This counterpressure pc(t) is usually controlled by a high-speed valve or valve system in conjunction with a pump. The relevant control of the valve or valve system is carried out by a control unit, which is preferably implemented using a microcomputer. The main input signals are the PPG signal v(t) and the cuff pressure pc(t). The pressure pc(t) required to keep the PPG signal v(t) constant now corresponds to the intra-arterial blood pressure pa(t).

For this it is necessary that the cuff pressure pc(t) can be changed at least as fast as the intra-arterial blood pressure pa(t) changes, so that the real-time condition is satisfied. The upper limit frequency of pa(t), and thus the highest rate of pressure change, is greater than at least 20 Hz, which is quite a challenge for a pressure control system. From this it follows that the pressure control using a valve or valve system is advantageously disposed in the immediate vicinity of the cuff. If the air lines are too long, there is a risk that this limit frequency condition will be lost due to the low-pass effect of the lines.

A mechanical valve known from U.S. Pat. No. 4,406,289 regulates the counterpressure in the finger cuff with the desired accuracy when it is supplied with a linearly operating pump. The valve is housed in a housing on the distal forearm and thus supplies the finger cuff with the pressure pc(t) via a short hose.

U.S. Pat. No. 4,524,777 describes a pressure generation system for the vascular unloading technique, wherein a constant cuff pressure Pc is also generated with a linear pump and is superimposed with pressure fluctuations Δpc(t) from a “shaker” or a “driving actuator” connected in parallel.

U.S. Pat. No. 4,726,382 discloses a finger cuff for the vascular unloading technique which has hose connections for supplying the cuff pressure pc(t). The length of the air hoses extends to the pressure generation system, which in turn is attached to the distal forearm.

WO 2000/059369 A1 also describes a pressure generation system for the vascular unloading technique. The valve system here comprises a separate inlet valve and a separate outlet valve. While a relatively linear proportional pump must be used in patent specifications U.S. Pat. No. 4,406,289 and U.S. Pat. No. 4,524,777, this system allows the use of simple, inexpensive pumps, since disruptive harmonics can be eliminated by the arrangement of the valves. Furthermore, the energy consumption of the simple pump can be significantly reduced by the valve principle.

A system for the vascular unloading technique is known from WO 2004/086963 A1 in which the blood pressure can be continuously determined in one finger, while the measurement quality is checked in the adjacent finger (“watch dog” function). After a period of time, the system automatically changes the “measuring finger” to the “monitoring finger”.

WO 2005/037097 A1 describes a control system for the vascular unloading technique having a number of intertwined control loops.

WO 2010/050798 A1 discloses a pressure generation system attached to the distal forearm (“front end”) and having only one valve, to which a finger cuff for the vascular unloading technique can be attached.

In a pressure generation system for the vascular unloading technique described in WO 2011/045138 A1—similar to that known from WO 2000/059369—the energy consumption of the pump is reduced and harmonics can be eliminated.

WO 2011/051819 A1 discloses an implementation of the vascular unloading technique that has been improved by means of digital electronics in order to increase stability and for further miniaturization.

WO 2011/051822 A1 describes a method for the vascular unloading technique in which the measured signals v(t) and pc(t) are processed to increase long-term stability and to determine other hemodynamic parameters. In particular, a method for eliminating effects originating from vasomotor changes in the finger arteries and a method for determining cardiac output (CO) are disclosed.

WO 2012/032413 A1 describes novel finger sensors that have a disposable part for single use. In this case, the cuff that comes into contact with the finger is accommodated in the disposable part for reasons of hygiene, whereas the associated pressure generation and pressure control system is accommodated in a reusable part. Accordingly, a separable pneumatic connection is to be provided here between the disposable part and the reusable part.

As a rule, the pressure generation and pressure control system in the prior art is attached to the distal forearm, proximal to the wrist, which has significant disadvantages: This location is often used for intravenous lines and the intra-arterial access at the distal end of the radial artery should also be free for emergencies. Such accesses can be blocked by the pressure generating and pressure control system and its attachment. In addition, the system can slip or tilt during operation. This can adversely affect how the sensors are seated. The seating of the sensors would also improve if the finger to be measured or the corresponding hand is in a certain rest position.

To overcome this problem, WO 2017/143366 A1 proposes a measuring system for the continuously determining the intra-arterial blood pressure on at least one finger of a hand, having at least one finger sensor, having a plethysmographic system, having at least one light source, preferably LED, having one or several wavelengths and at least one light sensor and at least one inflatable cuff, and having a pressure generation system with at least one valve controlled in real time using the plethysmographic system for generating a pressure in the cuff that is essentially equal to the intra-arterial blood pressure in the finger, wherein the measuring system has a housing with a surface that acts as a resting surface for the at least one finger and the adjacent regions of the palm. The hand rests on a resting surface under which are disposed essential components that were attached to the forearm in conventional systems.

Similar to WO 2012/032413 A1 mentioned above, the cuff is accommodated in a disposable part that can be separated from the housing (and thus from the resting surface). Correspondingly, a separable pneumatic connection between the disposable part and the reusable part is to be provided here, as well.

In the known systems, cuff pads, which are usually designed as soft-elastic cushions (made of PVC, for example) applied in a bent configuration or ring pads shaped like floating rings or doughnuts and which exert the (counter-)pressure on the finger inserted in the finger cuff, are connected to the pressure generation system via hose lines.

The production of such fluid, in particular air, supplies by means of hose lines, which are known from the prior art for pressurization, is costly and offers only limited possibilities for process automation and thus for cost-effective mass production. Furthermore, hose lines, especially their connection to the cuff pads, represent a challenge for quality assurance and quality control.

### DESCRIPTION OF THE INVENTION

In view of the limitations of conventional systems, it is an object of the present invention to improve cuff parts, in particular for measuring devices of the type mentioned above, especially with respect to aspects of their manufacture.

According to one aspect of the present invention, this object is achieved with a cuff part according to claim 1. Preferred embodiments of the invention can be implemented according to one of the dependent claims. In particular, the present invention thus provides a cuff part comprising at least two ring-like receiving tubes for receiving a portion of a respective finger of a hand passed through the respective receiving tube, at least one respective cuff pad arranged in each of the receiving tubes and fillable with a fluid, in particular air, and at least one respective fluid supply to the cuff pad, or cuff pads, respectively, of each of the receiving tubes, wherein the receiving tubes are formed in a shared receiving body, and the fluid supplies are formed as hose-free channels in the receiving body.

Advantageously for production, manufacturing processes that can be easily automated can be used, in particular (injection) molding and (CNC) milling processes.

According to an advantageous embodiment, the receiving body comprises a base body, which comprises recesses corresponding to the channels, and a cover, which is connected to the base body and delimits at least one of the channels from the outside. The cover can, for example, be welded or bonded to the base body. Advantageously suitable materials for the base body as well as for the cover can be, for example, ABS (acrylonitrile-butadiene-styrene), PC-ABS, SAN (styrene-acrylonitrile), PBT (polybutylene terephthalate), PC (polycarbonate), PS-HI (polystyrene), polyester (PET) as well as PVC. The base body can also be composed of two or more parts.

According to a, due to cost-effective practicability, particularly preferred embodiment, the base body may comprise an injection-molded part, in particular made of plastic.

In an advantageous enhancement of the invention, each of the cuff pads comprises a hard-elastic layer, which is inserted into the receiving tube and abutting the receiving body, and a soft-elastic layer, which is joined to the hard-elastic layer in an airtight manner at its edges (e.g. by welding, bonding or laminating together), for pressing against the respective finger, wherein the cuff pad can be filled with the fluid between the hard-elastic and the soft-elastic layer. Such a cuff pad can be manufactured, for example, like a soft blister pack known per se from the prior art. The layers can, but do not have to, each be multilayered. Plastic and composite materials known per se from the prior art can advantageously be suitable as layer materials, such as in particular PET (polyethylene terephthalate), PET-G for the hard-elastic layer and PUR (polyurethane), PVC (polyvinyl chloride), PA (polyamide) and coextrusion films (PA-PE, PP-PA-PE, PE-PET) for the soft-elastic layer. An inflow opening for the fluid can simply be punched out in the hard-elastic layer so that it aligns (or at least overlaps) with a corresponding opening of the channel of the associated fluid supply.

In the installed state in the respective receiving tube, the hard-elastic layer abutting the receiving body does not deform any further. The bulging of the preformed soft-elastic layer takes place by applying fluid pressure via the fluid supply.

Preferably, the hard-elastic layer is adhered to the respective receiving tube, for example by means of a two-sided adhesive tape or a hot-melt adhesive.

Preferably, for each of the channels, the cuff portion comprises a port associated with the respective channel for supplying the fluid into the cuff portion. In this way, the cuff pads can be pressurized with fluid pressure, in particular air pressure, independently of one another

According to an advantageous enhancement, the port can be a valve port. In this case, a valve device can be formed in the cuff part, or a valve function results from interaction with a fluid connection of an associated base part with the pressure supply. For example, a valve tappet and a spring-mounted closure body can interact here. A design having a valve connection is particularly suitable if the cuff part is interchangeable, in particular if it is designed as a disposable part. Interchangeable cuff parts are generally advantageous for adaptation to different hand or finger sizes and for hygienic reasons.

Preferably, the port comprises a sealing element that prevents fluid from escaping.

According to an advantageous further development, at least two of the ports can be arranged one behind the other with respect to the direction of intended insertion of the fingers into the receiving tubes, i.e. parallel to the axial direction of the receiving tubes, either in alignment or, if necessary, slightly offset. In this way, the channel arrangement can be implemented in a space-saving manner with regard to the required width of the cuff part. If the channels are arranged between the fingers, this avoids in particular the need to spread the fingers too far apart.

According to another aspect of the invention, there is provided a measuring device for parallel or alternating continuous determination of intra-arterial blood pressure on at least two fingers of a hand, comprising a base part, a cuff part according to one of the embodiments described above connectable to the base part without tools and disconnectable from the base part without tools, a radiation source for emitting light into the respective finger through an optical emission surface, a photodetector for detecting a portion of the light collected by an optical collector surface and not absorbed in the respective finger, and a pressure control system arranged at least partially in the base part and connectable to the ports of the cuff part for controlling a fluid pressure in the respective cuff pad in dependence on the detected non-absorbed portion of the light.

As explained above, an exchangeable design of the cuff part, especially as a disposable part, is generally advantageous for adaptation to different hand and finger sizes and for hygienic reasons.

In principle, every variant of the invention described or indicated in the context of the present application can be particularly advantageous, depending on the economic, technical and possibly medical conditions in the individual case. Unless stated otherwise, or as far as technically feasible in principle, individual features of the embodiments described are interchangeable or can be combined with one another as well as with features known per se from the prior art.

In particular, the technology used for pressure build-up in the cuff and for pressure regulation can basically be designed as known from the prior art.

The invention is explained in more detail herein-below in an exemplary manner with reference to the accompanying schematic drawings. The drawings are not to scale; in particular, for reasons of clarity, the proportions of the individual dimensions to one another sometimes do not correspond to the dimensional relationships in actual technical implementations. Corresponding elements are denoted by the same reference signs in the drawings.

### BRIEF DESCRIPTION OF THE DRAWINGS

FIG. 1 shows a measuring device according to the invention in a side view, wherein a cuff part comprising an ergonomic hand rest and a finger-receiving part is placed on a base part containing a pressure control system,

FIG. 2 shows a perspective view of the measuring device of FIG. 1 from obliquely above,

FIG. 3 shows a perspective view of the measuring device of FIG. 1 from obliquely below,

FIG. 4 shows a cuff part like the one shown in FIG. 3 from obliquely below, but without the associated base part, the cuff pads not being shown,

FIG. 5a shows a view of the measuring device from FIG. 1 from behind, i.e. from the side of the palm rest (from the left in FIG. 1),

FIG. 5b shows a sectional view of a section of the cuff part of the measuring device shown in FIG. 5a, the sectional plane is indicated by the broken line A-A′ in the top view of FIG. 6a of the cuff part,

FIG. 5c shows a sectional view of a section of the cuff part of the measuring device shown in FIG. 5a, the sectional plane parallel to FIG. 5b is indicated by the broken line B-B′ in the top view of FIG. 6a on the cuff part,

FIG. 6a shows the cuff part of the device of FIGS. 5a-5c in plan view without cover, with the position of the channels and ports made visible relative to each other,

FIG. 6b shows the cuff part of the device from FIGS. 5a-5c in plan view with cover delimiting the channels from the outside,

FIG. 7 shows a possible valve device for the ports for fluid supply, wherein the valve device is shown once in the open and once in the closed state,

FIG. 8a shows a cuff pad before installation, with the viewing direction pointing obliquely towards the bulged soft elastic layer,

FIG. 8b shows the cuff pad from FIG. 8a bent as in the installation position, with the viewing direction pointing obliquely towards the bulged soft elastic layer,

FIG. 8c shows the still unbent cuff pad from FIG. 8a, with the viewing direction pointing obliquely towards the hard-elastic layer,

FIG. 8d shows the bent cuff pad of FIG. 8b, with the viewing direction pointing obliquely towards the hard-elastic layer

The blood pressure measuring device 1 is designed as a photoplethysmographic measuring system which functions according to the so-called “Vascular Unloading Technique”. Measuring components, i.e. in particular optical and electronic components as well as mechanical components of the pressure generation and pressure control system accommodated in the base part 3, can be implemented in principle in a manner similar to the prior art mentioned at the beginning. The cuff part placed on the base part 3 comprises an ergonomic palm rest 4, an ergonomic finger rest 6 divided by a web 5, and the receiving part 7.

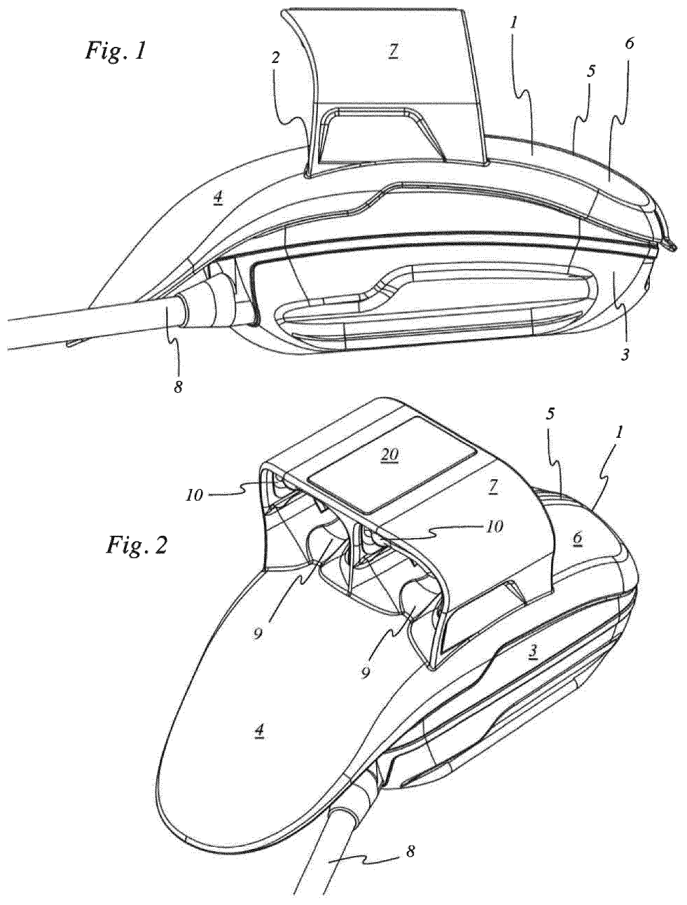

As shown in the perspective view of FIG. 2 (viewed from obliquely above at the right in FIG. 1), the receiving part 7 is designed to receive two fingers, which makes alternate measurement on both fingers possible. For reasons of hygiene, the cuff part 2, together with the palm rest 4 and finger rest 6, is designed as a disposable article which is detachably, without tools, attached to the reusable base part 3 by means of a plug-in connection.

The pressure control system in the base part 3 is supplied with compressed air via the obliquely laterally attached cable 8, which points in the direction of the forearm when the hand is placed on the base part as intended. Furthermore, the cable 8 serves to supply energy to the pressure control system (not shown), light sources (not shown) and photodetectors (not shown) or associated control, amplifier and evaluation circuits in the measuring device 1. Measurement data can be output to a patient monitor via a suitable electronic interface through the cable 8.

The lateral attachment of the cable 8, pointing in the direction of the forearm when the hand is placed as intended, has the advantage that the cable can be guided along the patient's arm, but the wrist area with tendons and vessels does not rub against the cable 8, and in particular the carpal tunnel is also protected.

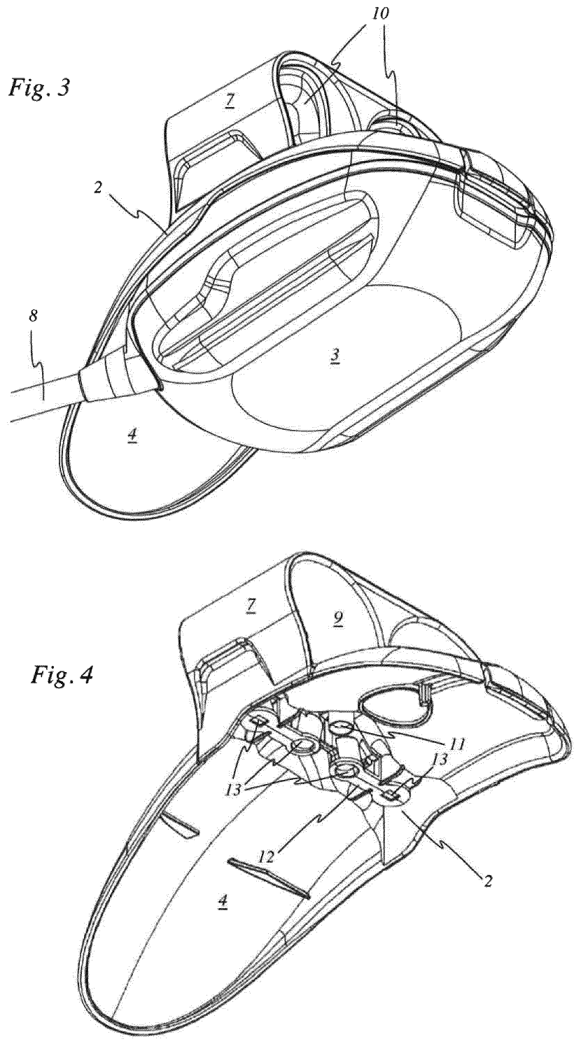

The two inflatable cuff pads 10 arranged in the receiving tubes 9 are each connected to the pressure generation and pressure control system via a port 11 at the interface between cuff part 2 and base part 3. In the illustration of cuff part 2 in FIG. 4, one of the two ports 11 is hidden by the crosspiece 12, which carries light guides 13 through which light is conducted from the light sources arranged in base part 3 to the fingers or from the fingers to the photosensors arranged in base part 3.

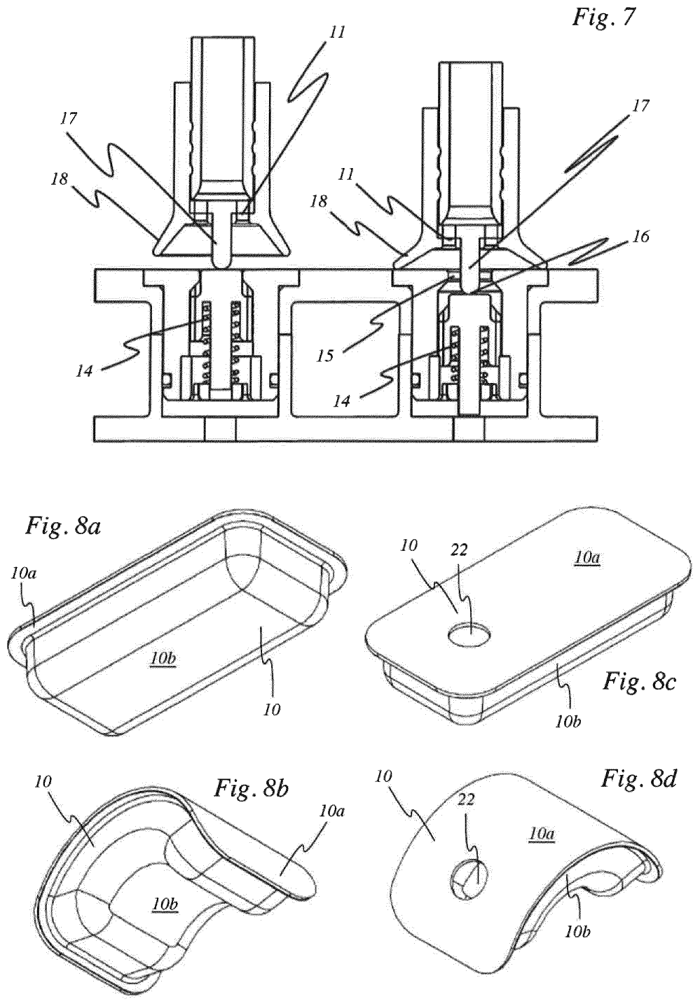

A valve device is preferably located at port 11, so that port 11 is flush with the housing of base part 3 on the base part side when base part 3 and cuff part 2 are not connected to each other. Such a valve device is exemplarily shown in FIG. 7. On the base part side, the fluid passage 15 is closed by the valve body 16 spring-mounted by means of the spring 14. The port 11 of the cuff part 2 comprises a valve tappet 17 which, in the connected state, presses the valve body 16 downward to release the fluid passage 15. Sealing to the outside is provided by a sealing element 18 designed as an elastic conical collar.

The channels 19, which connect the respective port 11 to the respective cuff pad 20, are formed in the base body 21 of the receiving body 7, which is made of plastic by means of injection molding, and are delimited from the outside by means of the plastic cover 20, which is welded or bonded on.

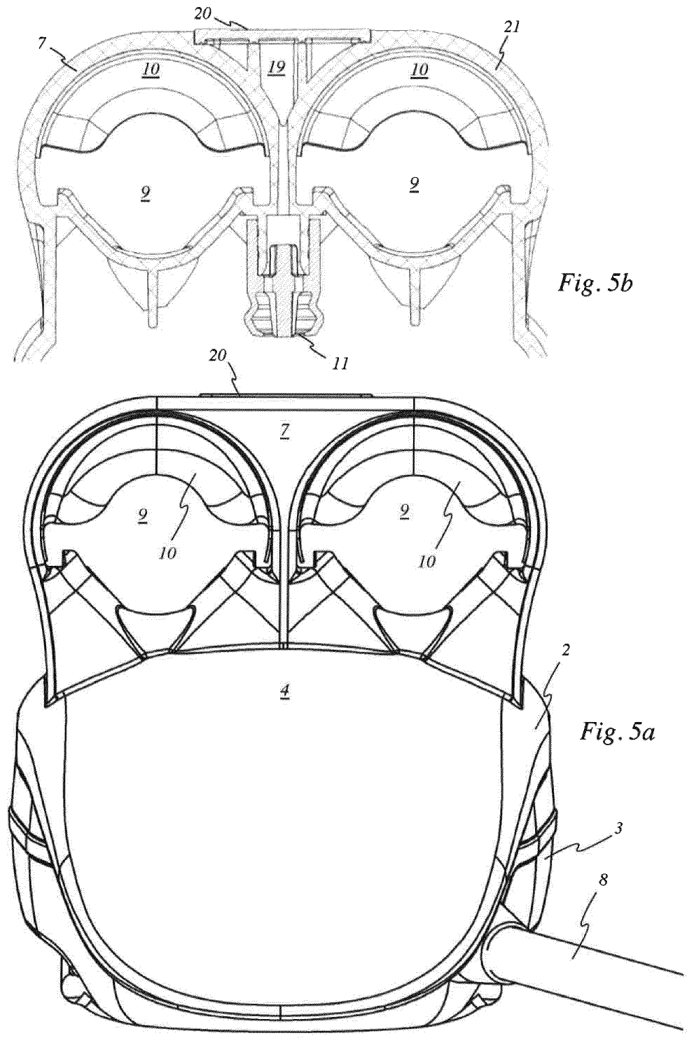

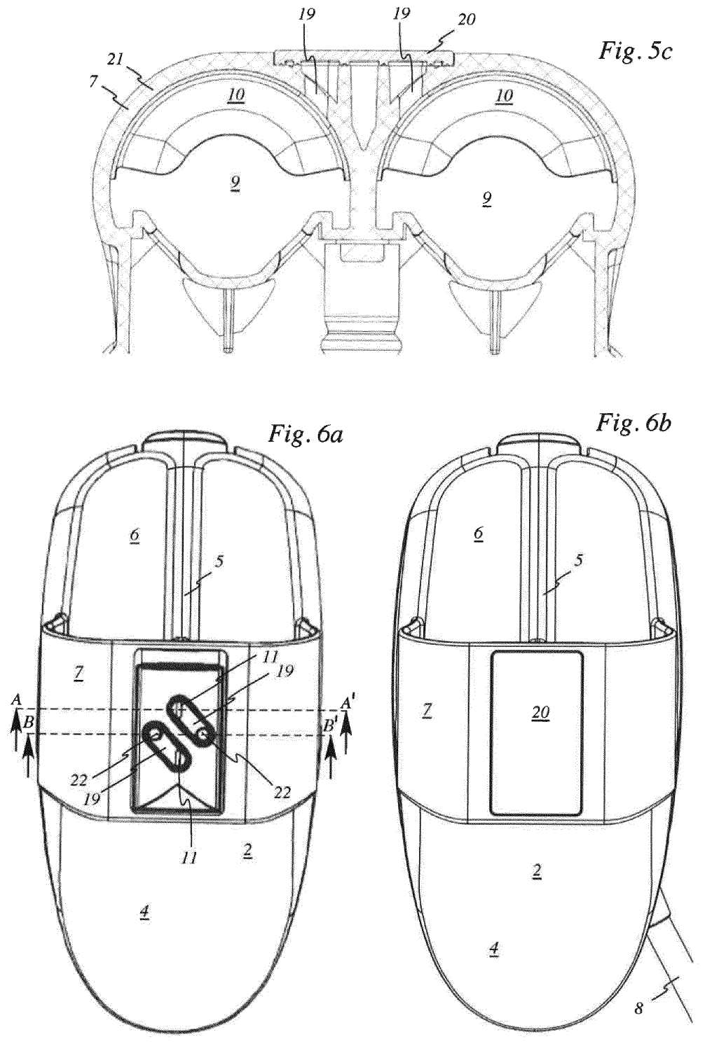

The layout of the channels 19 in the base body 21 of the receiving body 7 is illustrated by the sectional views in FIGS. 5b and 5c together with the plan view of FIG. 6a. For orientation, the sectional plane of FIG. 5b is shown as broken line A-A′ and the sectional plane of FIG. 5c is shown as broken line B-B′ in FIG. 6a.

The ports 11 are situated one behind the other in a direction parallel to the axial direction of the receiving tubes 9. From there, the respective channel 19 is first routed upward, as can be seen in FIG. 5b, further diagonally to the side, as shown in FIG. 6a, and finally downward to the fluid access opening 22 of the respective cuff pad 10, as can be seen in FIG. 5c.

As can be seen in FIGS. 8c and 8d, the fluid inlet opening 22 is punched into the hard-elastic layer 10a of the cuff pad 10, which is bonded to the receiving body 7 for mounting in the respective receiving tube 9. The preformed soft-elastic layer 10b is connected to the hard-elastic layer 10a in an airtight manner at its edges, so that the space between the layers 10a, 10b can be filled with fluid, in particular air, and pressurized.

In FIGS. 8b and 8d, the cuff pad is shown as bent in the installed position, and in FIGS. 8a and 8c it is shown unbent before installation for illustrative purposes.

1. A cuff part comprising
at least two ring-like receiving tubes for receiving a portion of a respective finger of a hand passed through the respective receiving tube,
at least one fluid-fillable cuff pad disposed in each of the receiving tubes, and
at least one respective fluid supply to the cuff pad or pads of each of the receiving tubes,
said receiving tubes being formed in a shared receiving body, and
said fluid supplies are formed as hose-free channels in said receiving body.

2. The cuff part according to claim 1, wherein the receiving body comprises a base body comprising recesses corresponding to the channels, and a cover connected to the base body and delimiting at least one of the channels from the outside.

3. The cuff part according to claim 2, wherein the base body comprises an injection-molded part.

4. The cuff part according to any one of the preceding claims, wherein each of the cuff pads comprises a hard-elastic layer inserted into the receiving tube and abutting the receiving body, and a soft-elastic layer for pressing against the respective finger, said soft elastic layer being connected at its edges to the hard-elastic layer, the cuff pad being fillable with the fluid between the hard-elastic and soft-elastic layers.

5. The cuff part according to claim 4, wherein the hard-elastic layer is adhered into the respective receiving tube.

6. The cuff part according to any one of the preceding claims, further comprising for each of the channels a port associated with the respective channel for supplying the fluid into the cuff part.

7. The cuff part according to claim 6, wherein the port is a valve port.

8. The cuff part according to claim 6 or claim 7, wherein the port comprises a sealing element.

9. The cuff part according to any one of claims 6 to 8, wherein at least two of the ports are arranged one behind the other, offset or aligned, with respect to the direction of intended insertion of the fingers into the receiving tubes.

10. A measuring device for parallel or alternating continuous determination of the intra-arterial blood pressure on at least two fingers of a hand, which comprises:
a base part,
a cuff part according to any of claims 6-8, which is connectable to the base part without tools and separatable from the base part without tools,
a radiation source for emitting light into the respective finger through an optical emission surface,
a photodetector for detecting a portion of the light collected by an optical collector surface and not absorbed in the respective finger, and
a pressure control system arranged at least partially in the base part and connectable to the ports of the cuff part for controlling a fluid pressure in the respective cuff pad depending on the detected non-absorbed portion of the light.
